(19) United States
(12) Patent Application Publication
(10) Pub. No.: US 2010/0152670 A1
(43) Pub. Date: Jun. 17, 2010

(54) SAFETY NEEDLE CAP FOR SYRINGE

(75) Inventor: Tzu-Yi LOW, Taipei City (TW)

Correspondence Address: BRIAN M. MCINNIS, 12th Floor, Ruttonjee House, 11 Duddell Street, Hong Kong, HK

(73) Assignee: Tzu-Yi LOW, TAIPEI (TW)

(21) Appl. No.: 12/336,549

(22) Filed: Dec. 17, 2008

(51) Int. Cl. A61M 5/32

(52) U.S. Cl. 604/198

(57) ABSTRACT

A safety needle cap for enclosing a needle assembly of a syringe therein includes a tubular cap body having opposite open end and closed end. The cap body is internally provided near the open end with a plurality of radially inward protruded ribs, and is formed on one side with a slit extended from the open end in a length direction of the cap body, so that the needle assembly can be set in the cap body via the slit in a cross sectional direction of the needle assembly. Therefore, a user is protected against stabbing by a sharp tip of the needle assembly. A length of the slit near the open end of the cap body has two outward expanded lateral edges to provide a flared cross section thereat, enabling the needle assembly to be more easily set in the safety needle cap via the slit.

### FIELD OF THE INVENTION

The present invention relates to a tubular cap, and more particularly to a safety needle cap having a tubular cap body.

### BACKGROUND OF THE INVENTION

A syringe is a device for injecting a liquid substance, such as a medical liquid, blood, a nutrient solution, etc., into a patient's body. The syringe includes a cannula for inserting into the patient's body, and a plunger for pushing the liquid substance stored in a barrel into the patient's body via the cannula.

Since the cannula inserted into the patient's body in the injection has contacted with the patient's blood, the used cannula must be properly disposed after the injection to avoid contact of the used cannula with any other people to cause undesirable infection.

For this purpose, there is developed a safety syringe, with which the used cannula thereof is received or broken in a container, preventing other people from unexpectedly contacting with the used cannula and becoming infected.

FIGS. 1 and 2 are assembled and exploded perspective views, respectively, of a conventional safety syringe.

The conventional safety syringe includes a barrel assembly 1, a needle assembly 2, and a tubular needle cap 3. The barrel assembly 1 includes a barrel body 11 and a plunger 12. The barrel body 11 has opposite first end 111 and second end 112. The second end 112 of the barrel body 11 is provided with an internally threaded bore 113. The plunger 12 is fitted into the barrel body 11 via the first end 111 thereof. The needle assembly 2 includes a cannula 21 and a needle hub 22 for supporting the cannula 21 on a front end thereof. The needle hub 22 is provided on an outer periphery with a plurality of circumferentially spaced and radially outward protruded ribs 221. The needle hub 22 is provided at a rear end with two diametrically opposite and radially outward projected flanges 222. The flanges 222 serve as male threads for screwing into the internally threaded bore 113 on the barrel assembly 1, so that the needle assembly 2 and the barrel assembly 1 are connected to each other and form an integral unit. The needle cap 3 includes a tubular cap body 31 having opposite open end 311 and closed end 312. The cap body 31 is internally provided near the open end 311 with a plurality of radially inward protruded ribs (not shown). The needle assembly 2 is inserted into and held in the needle cap 3 via the open end 311, so that the needle cap 3 encloses and isolates the needle assembly 2 from external environment.

To use the syringe, first remove the needle cap 3 from the needle assembly 2 to expose the latter. Then, the plunger 12 can be pulled or pushed along inside the barrel body 11, allowing the syringe to take in or expel a necessary liquid via the needle assembly 2. When the liquid taken in has been injected into a patient's body via the cannula 21, the needle cap 3 is covered onto the entire needle assembly 2 again and turned. At this point, the inward ribs in the needle cap 3 will abut on the outward ribs 221 on the outer periphery of the needle assembly 2 to drive the needle assembly 2 to rotate at the same time and accordingly be screwed off from the barrel assembly 1. The needle assembly 2 separated from the barrel assembly 1 is received in the needle cap 3 to form an integral unit, which is then disposed as a contaminant. The barrel assembly 1 that did not contact with the patient in the injection can be separately disposed or properly recycled.

The conventional safety syringe is simple in structure and convenient for use, and can be produced at low cost, and is therefore widely adopted in medical field. However, the conventional safety syringe also has the following disadvantage: when the injection has been completed and a nurse wants to cover the used needle assembly 2 with the needle cap 3 again, the nurse would have to forward insert the needle assembly 2 through the open end 311 into the needle cap 3 in a length direction of the needle assembly 2. By doing this, any negligent operation of the nurse or any unexpected impact on the nurse would bias the pointed end of the cannula 21 from the open end 311 of the needle cap 3 to stab into the nurse's hand and cause undesirable infection to the nurse.

### SUMMARY OF THE INVENTION

It is therefore a primary object of the present invention to provide a safety needle cap capable of protecting a user against the risk of being unexpectedly stabbed and accordingly contaminated by a used needle of a syringe.

To achieve the above and other objects, the safety needle cap according to the present invention is designed for a user to safely enclosing a needle assembly of a syringe therein. The safety needle cap includes a tubular cap body having an open end and a closed end opposite to the open end. The cap body is internally provided near the open end with a plurality of circumferentially spaced and radially inward protruded ribs. The cap body is provided on one side with a slit extended from the open end in a length of the cap body, so that the needle assembly of the syringe can be downward set in the cap body via the slit in a cross sectional direction of the needle assembly without being forward pushed to dangerously stab the user.

Preferably, a length of the slit near the open end of the cap body has two outward expanded lateral edges, so that the slit at that portion has a flared cross section, enabling the cannula of the needle assembly to be more easily set in the cap body via the slit.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein

FIG. 1 is an assembled perspective view of a conventional safety syringe;

FIG. 2 is an exploded view of the conventional safety syringe of FIG. 1;

FIG. 3 is a perspective view showing a safety needle cap according to a preferred embodiment of the present invention and a syringe with which the safety needle cap is used; wherein a needle assembly and a barrel assembly of the syringe are separated from each other;

FIG. 4 shows the syringe of FIG. 3 with the needle assembly and the barrel assembly thereof in an assembled state, and the safety needle cap of the present invention before being covered on the needle assembly;

FIG. 5 shows the manner of approaching the needle assembly of the syringe to the safety needle cap of the present invention to safely set the needle assembly in the safety needle cap;

FIG. 6 is a longitudinal sectional view showing the safety needle cap of the present invention with the needle assembly of the syringe set therein;

FIG. 7 is a cross sectional view taken along line A-A′ of FIG. 6; and

FIG. 8 shows the safety needle cap of the present invention along with the needle assembly are screwed off from the barrel assembly of the syringe with the needle assembly received in the safety needle cap.

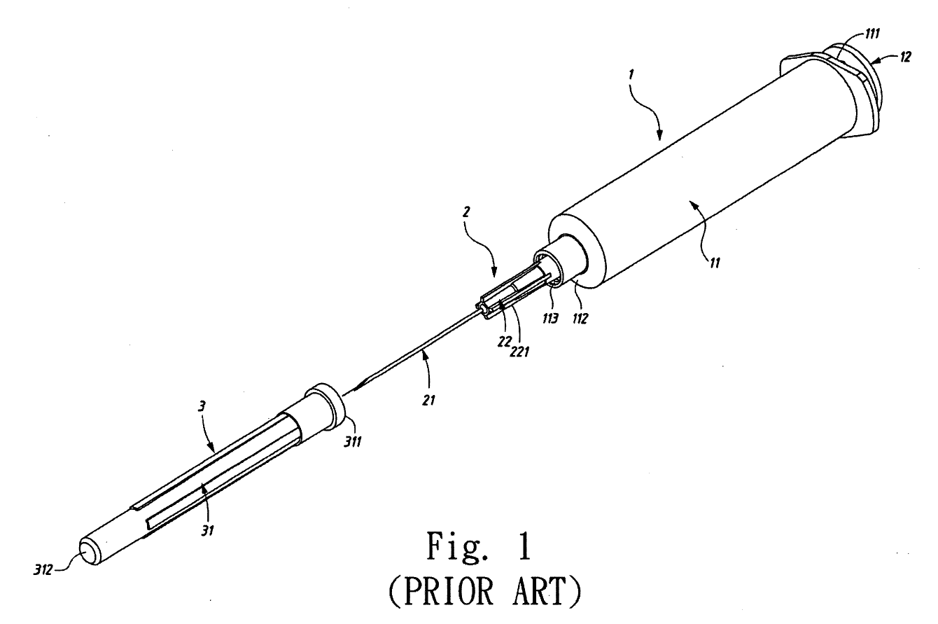

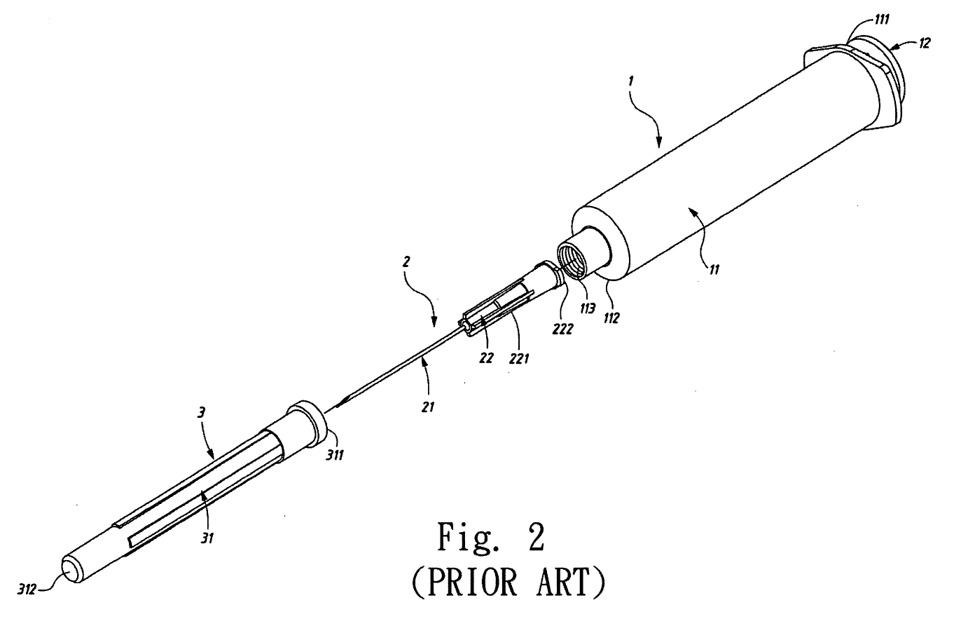

### DETAILED DESCRIPTION OF THE PREFERRED EMBODIMENTS

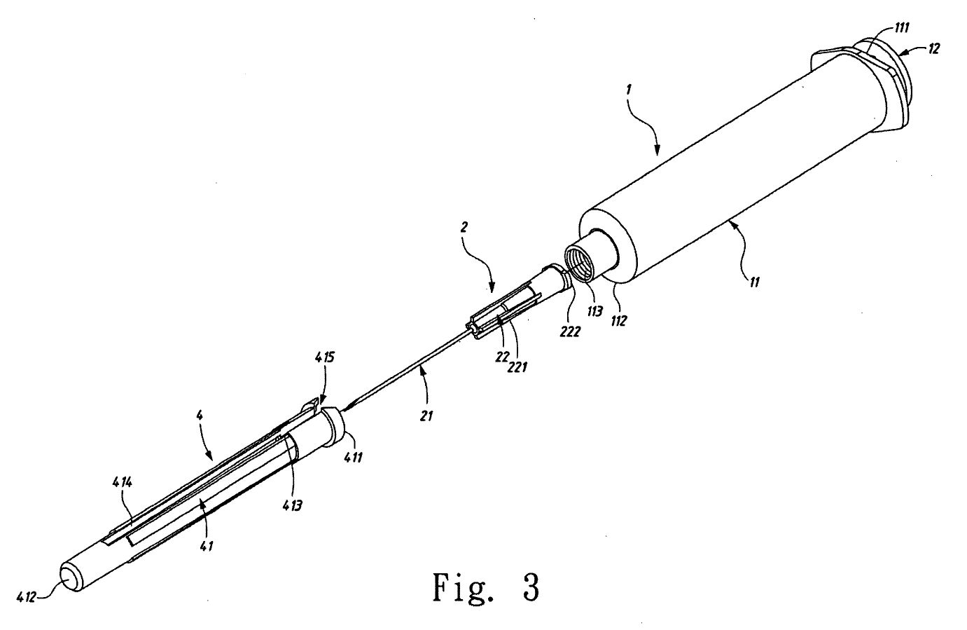

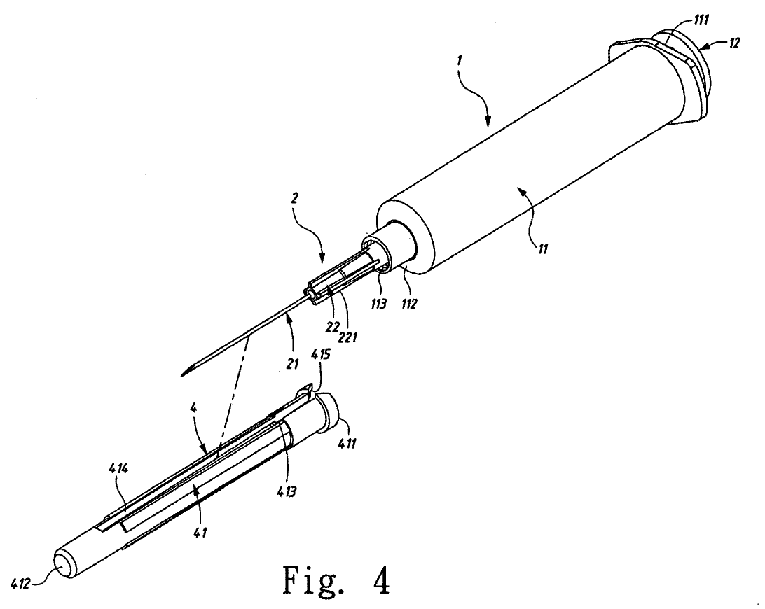

Please refer to FIG. 3 that is a perspective view showing a safety needle cap 4 according to a preferred embodiment of the present invention and a syringe with which the safety needle cap 4 is used, wherein a needle assembly 2 and a barrel assembly 1 of the syringe are separated from each other; and to FIG. 4 that shows the syringe of FIG. 3 with the needle assembly 2 and the barrel assembly 1 thereof in an assembled state, and the safety needle cap 4 of the present invention before being covered on the needle assembly 2.

As shown, the safety needle cap 4 of the present invention is different from the conventional needle cap 3 in that a slit 414 is provided on one side of a tubular cap body 41 of the safety needle cap 4 to extend in a length direction of the cap body 41, so that the needle assembly 2 can be safely set in the safety needle cap 4 via the slit 414. More specifically, the safety needle cap 4 of the present invention includes a tubular cap body 41, which has an open end 411 and a closed end 412 opposite to the open end 411. The cap body 41 is internally provided near the open end 411 with a plurality of circumferentially spaced and radially inward protruded ribs 413. And, the cap body 41 is further provided on one side thereof with a slit 414 of a predetermined length, which extends from the open end 411 in a length direction of the cap body 41.

The safety needle cap 4 of the present invention is so designed that it can be used with the barrel assembly 1 and the needle assembly 2 of a conventional safety syringe. The barrel assembly 1 and the needle assembly 2 have conventionally known structures. That is, the barrel assembly 1 includes a barrel body 11 and a plunger 12. The barrel body 11 has a first end 111 and a second end 112 opposite to the first end 111, and the second end 112 is provided with an internally threaded bore 113. The plunger 12 is fitly received in the barrel body 11 via the first end 111 thereof. The needle assembly 2 includes a cannula 21 and a needle hub 22 for supporting the cannula 21 thereon. The needle hub 22 is provided on a front outer periphery thereof with a plurality of circumferentially spaced and radially outward protruded ribs 221, and on a rear end at two diametrically opposite sides with two spaced and radially outward flanges 222. The two flanges 222 can serve as external threads to mesh with the internally threaded bore 113, so that the needle hub 22 can be screwed into the barrel assembly 1 via the internally threaded bore 113 to thereby assemble the needle assembly 2 to the barrel assembly 1.

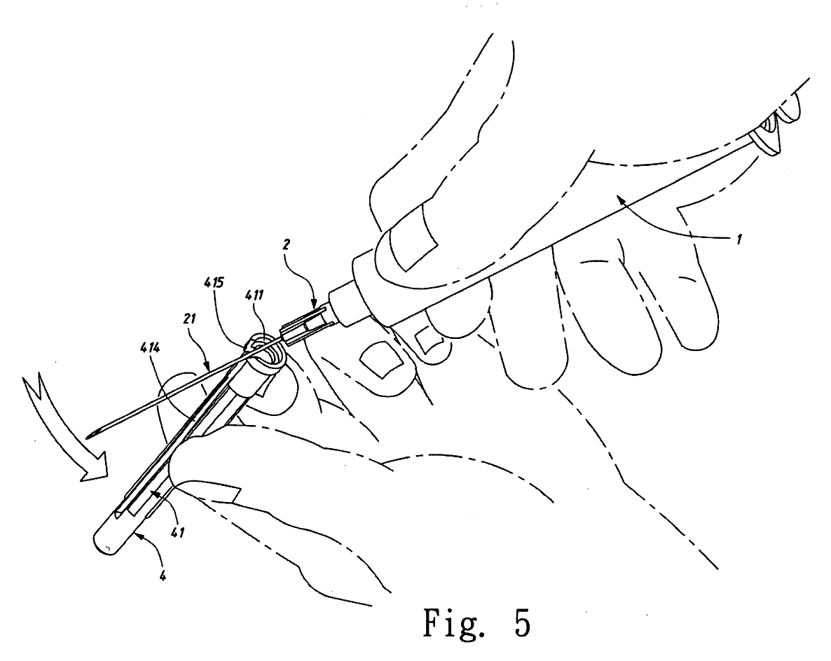

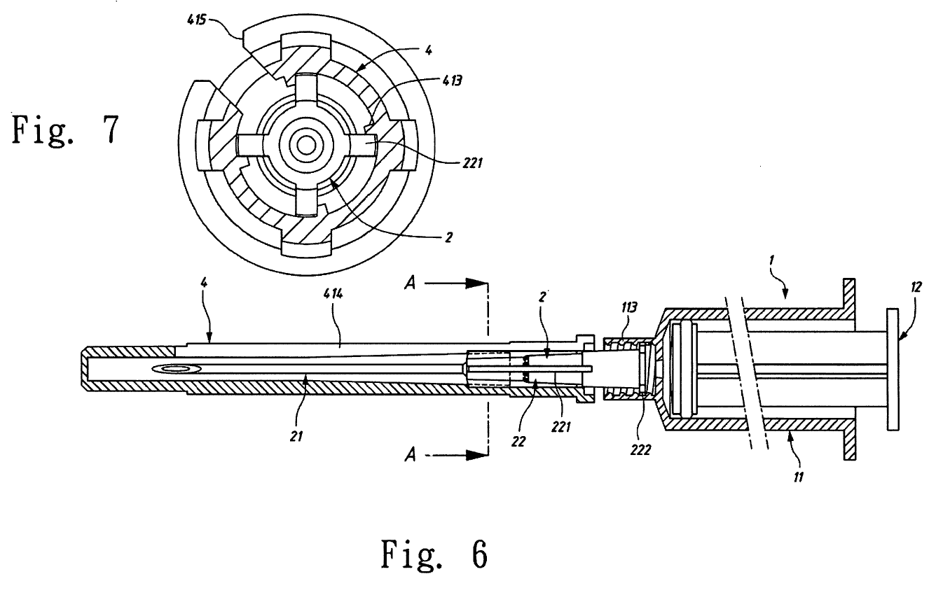

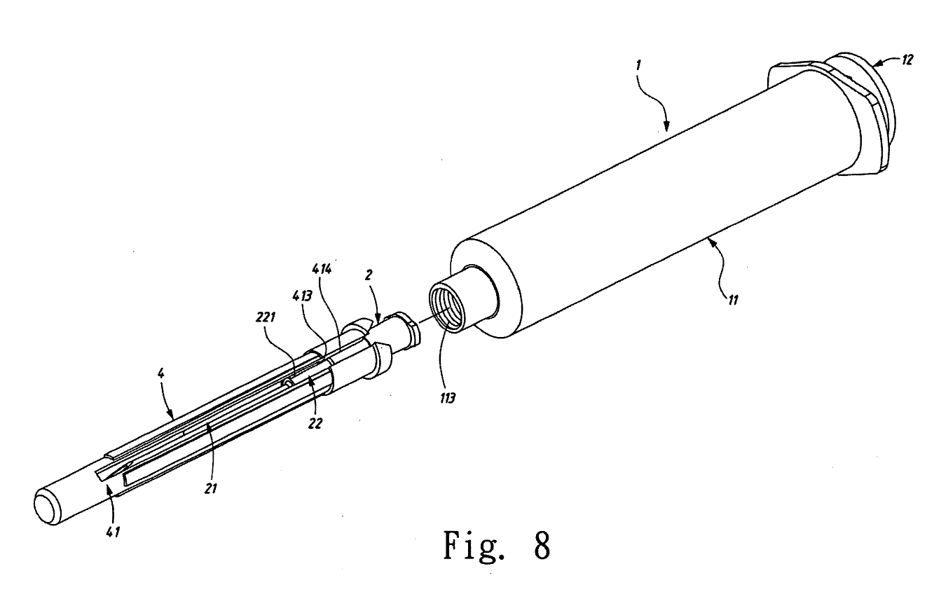

Please refer to FIGS. 5 to 8. FIG. 5 shows the manner of approaching the needle assembly 2 of the syringe to the safety needle cap 4 of the present invention to set the needle assembly 2 in the safety needle cap 4; FIG. 6 is a longitudinal sectional view showing the safety needle cap 4 of the present invention with the needle assembly 2 of the syringe set therein; FIG. 7 is a cross sectional view taken along line A-A′ of FIG. 6; and FIG. 8 shows the safety needle cap 4 of the present invention along with the needle assembly 2 are screwed off from the barrel assembly 1 of the syringe with the cannula 21 of the needle assembly 2 set in the safety needle cap 4.

When a nurse inserts the cannula 21 into a patient's body and completes an injection, the used needle assembly 2 is then to be received in the safety needle cap 4. To do so, the nurse can downward approach the needle assembly 2 to the safety needle cap 4 in a cross sectional direction of the cannula 21, so that the cannula 21 is set in the safety needle cap 4 via the slit 414 and the needle assembly is finally axially laid in the safety needle cap 4, as shown in FIGS. 4, 5 and 6. Then, the safety needle cap 4 can be turned. At this point, as shown in FIG. 7, the inward ribs 413 in the safety needle cap 4 will abut on the outward ribs 221 on the needle hub 22 to drive the needle assembly 2 to rotate along with the safety needle cap 4, so that the needle assembly 2 is screwed off from the barrel assembly 1, as shown in FIG. 8. The needle assembly 2 having been screwed off from the barrel assembly 1 is set in the safety needle cap 4 as an integral unit and can be then disposed as a contaminant. The barrel assembly 1 that did not contact with the patient can be separately disposed or properly recycled.

With the safety needle cap 4, the needle assembly 2 is not inserted into the safety needle cap 4 in a length direction of the cannula 21. That is, the needle assembly 2 is not inserted into the safety needle cap 4 by pointing a sharp tip of the cannula 21 of the needle assembly 2 toward the nurse's hand in a direction of applying an insertion force. Instead, the needle assembly 2 is downward approached to the safety needle cap 4 in a cross sectional direction of the cannula 21, so that the cannula 21 is set in the safety needle cap 4 via the slit 414. Therefore, the nurse can be protected from being stabbed by a forward pushed sharp tip of the cannula 21 to avoid the risk of being contaminated.

The slit 414 has a small width only slightly larger than a thickness of the cannula 21. With this small width, it is impossible for a user's finger to touch the cannula 21 via the slit 414. Therefore, the safety needle cap 4 with the slit 414 is safe for use to ensure the user's safety against contamination.

For the user to more easily set the cannula 21 in the safety needle cap 4 via the small slit 414 from the very beginning, a small length of the slit 414 adjacent to the open end 411 has two outward expanded lateral edges to provide a flared cross section 415 as can be seen in FIG. 7.

The present invention allows a user to downwardly set the cannula 21 in the safety needle cap 4 to avoid the risk of being stabbed and becoming contaminated by the cannula 21. Therefore, the safety needle cap 4 is convenient and safe for use and has good applicability.

The present invention has been described with a preferred embodiment thereof and it is understood that many changes and modifications in the described embodiment can be carried out without departing from the scope and the spirit of the invention that is intended to be limited only by the appended claims.

What is claimed is:

1. A safety needle cap for enclosing a needle assembly of a syringe therein, comprising a tubular cap body, the tubular cap body having an open end and a closed end opposite to the open end, and being internally provided near the open end with a plurality of circumferentially spaced and radially inward protruded ribs; the safety needle cap being characterized in that the tubular cap body is provided on one side with a slit, which is extended from the open end in a length direction the cap body, so that the needle assembly of the syringe can be set in the cap body via the slit in a cross sectional direction of the needle assembly.

2. The safety needle cap as claimed in claim 1, wherein a length of the slit near the open end of the cap body has two outward expanded lateral edge to provide a flared cross section thereat.
